(19) United States
(12) Patent Application Publication
(10) Pub. No.: US 2003/0129190 A1
(43) Pub. Date: Jul. 10, 2003

(54) FX activity in cells in cancer, inflammatory responses and diseases and in autoimmunity

(75) Inventors: Isaac P. Witz, Tel Aviv (IL); Rinat Eshel, Tel Aviv (IL)

Correspondence Address: Gary M. Nath, NATH & ASSOCIATES PLLC, 6th Floor, 1030 15th Street, N.W., Washington, DC, 20005, US

(73) Assignee: Ramot University Authority for Applied Research & Industrial Development Ltd., Tel Aviv (IL)

(21) Appl. No.: 10/342,323

(22) Filed: Jan. 15, 2003

Related U.S. Application Data
(63) Continuation of application No. 09/456,540, filed on Dec. 8, 1999, now abandoned.

(51) Int. Cl. A61K 39/395

(52) U.S. Cl. 424/146.1; 424/155.1

(57) ABSTRACT

A method is provided for inhibiting or preventing adherence of cancer cells or immunocytes to their target cells or target tissues by reducing the level of activity of FX enzyme in the cells. The reduction may either be in the amount of the FX protein or in the enzymatic activity of the protein in the cells.

In addition, methods and agents are provided for down-regulating or up-regulating the level of activity of FX in cancer cells and in immunocytes. The down-regulation of FX in cancer cells is provided for inhibiting or preventing metastasis of cancer cells and the up-regulation of FX activity in cancer cells is useful for increasing the immunogenicity of these cells when used in a cancer vaccine. Down-regulation of FX in immunocytes inhibits or prevents conditions such as undesired inflammatory responses, auto-immunity and transplant rejection. Up-regulation of FX activity in immunocytes is useful for enhancing an immune reaction in cases of reduced immunity of an individual.

The regulation of the level of activity of FX in cancer cells and immunocytes may be achieved by contacting the cells with various agents such as antibodies, growth factors, cytokines or various combinations of such agents.

The level of activity of FX in cancer cells is also used as a diagnostic and prognostic factor of metastasis and the level of activity of FX in immunocytes is used as a marker of activation useful in determining an individual's response to an immunogen.

### FIELD OF THE INVENTION

The present invention is generally in the field of diagnosis and therapy of cancer and of inflammatory diseases and of autoimmune diseases. The invention concerns compositions and methods for reducing the adherence of malignant and inflammatory cells to target tissue and endothelium.

### LIST OF PRIOR ART

The following is a list of references which are intended for better understanding of the present invention:

Brakenhoff, R. H., et al, J. Cell Biol., 129:1677-1689 (1995)

De Bree, R., Roos, J. C., Qauk, J. J., Den Hollander, W., Wilhelm, A. J., Van Lingen, A., Snow, G. B. and Van Dongen, G. A. M. S., Clin. Cancer Res. 1:227 (1995).

Fuhlbrigge, R. C., Alon, R., Puri, K. D., Lowe, J. B., and Spinger, T. A., J. Cell. Biol., 135:837-848 (1996).

Gerretsen, M., Visser, G. W. M., Brakenhoff, R. H., Van Walsum, M., Snow G. B. and Van Dongen, G. A. M. S., Cell Bioph., 24:135 (1994).

Hansen, H. J., Goldenberg, D. M., Newman, E. S., Grebenau, R., and Sharkey, R. M., Cancer, 71:3478-3485 (1993).

Kniep, B., Peter-Katalinic, J., Muthing, O., Pickl, W. F., and Knapp, W., J. Biochem. (Tokyo), 119:456-462 (1996).

Ito, H., et al, Int. J. Cancer, 71:556-564 (1997).

Kannagi, R., Glycoconj. J., 14:557 (1997).

Kong, R. K. M., Barrios, A., Knapp, W., and Macher, B. A. Arch. Biochem. Biophys., 300:677 (1993).

Kurahara Shin-Ichi et al., Carbohydrate Antigens in Oral Cancer, 330:, (1999)

Laemmli, U. K., Nature (Lond.) 227:680-685 (1970).

Lawrence, M. B., and Springer, T. A., Cell, 65:859-873 (1991).

Liang, P., and Pardee, A. B., Science, 257:967-971 (1992).

Quak, J. J., Bahn, A. J. M., Van Dongen, G.AMS., Brakkee, J. G. P., Scheper, R. J., Snow, G. B. and Meijer, C. J. L. M., Am. J. Pathol. 136:191 (1990).

Sugita, Y., and Masuho, Y., Immunotechnology, 3:157-168 (1995).

Szikora, Jean-Pierre et al., The EMBO Journal, 9:1041-1050, (1990).

Toneni-Michela; Sturla-Laura; Bisso-Angela; Benatti-Umberto; De-Flora-Antonio, Journal of Biological Chemistry, 271(44): 27274-27279 (1996).

Walz, G., Aruffo, A., Kolanus, W., Bevilacqua, M., and Seed, B., Science 250:1132 (1990).

Glossary

In the following description and claims use will be made, at times, with a variety of terms and the meaning of such terms as they should be construed in accordance with the invention is as follows:

Level of activity—refers to one of the following possibilities:

(i) Where the level of activity is affected by the amount of the enzyme. The alterations in the amount of FX may be at the level of the protein itself (i.e. by its degradation). This may be measured, for example, by preparing immunoblots in which the antibody is an anti FX antibody. In addition, the altering in the amount of FX may be at the level of its expression, i.e. inhibition of transcription of a genetic sequence, inhibition of translation of an mRNA sequence, etc. This may then be measured, for example, by preparing Southern or Northern blots as known in the art.

(ii) Where the amount of the enzyme remains at a constant level but its enzymatic activity is changed. This may be due to a reduction or elevation of the activity of the protein itself (i.e. by lack of an essential co-factor, lack of an appropriate substrate, inhibitors, agonists, etc.). Such alterations in the activity of FX may be determined by any of the assays known in the art such as in vitro assays of epimerase and reductase activities described, for example, in Sullivan, F. X., et al, J. Biol. Chem., 273:8193, (1998). Alternatively, the changes in the activity of the enzyme may be due to an inhibition of its expression either during transcription of a DNA sequence or during translation of a mRNA sequence., This may be achieved, for example, by the use of antisense sequences, a single chain anti FX antibody sequence, ribozymes etc.

Reduction—In accordance with the aspect of the invention relating to the metastatic process, this term should be construed as meaning a level of activity of FX in the cells which is within the limits of the activity of FX in non malignant cells. In accordance with the aspect of the invention relating to the inflammation, auto-immune and transplant rejection aspect, the reduction is to a level of activity of FX which is within the limits of the activity of FX in non activated immunocytes.

FX—is the enzyme (GDP-keto-6-dioximannose-3, 5-epimerase, 4-reductase) which catalyzes a combined epimerase and reductase reaction which converts GDP-4-keto-6-D-dioximannose to GDP-L-fucose, or a fragment or derivative of said enzyme which essentially maintains the activity of FX.

Cancer cells—are cells which are able to grow in culture in vitro and/or to form tumors when injected in vivo. The cancer cells may originate from any kind of cancer including solid tumors such as, for example, squamous cell carcinoma (SCC), colon carcinoma, etc., or non-solid tumors such as, for example, leukemia.

Effective amount—relates to an amount of the active agent which upon contact with the cells results in reduction or enhancement of activity of FX as the case may be. The effective amount depends on various parameters including, for example, the type of agent, whether it exerts its effect extracellularly or intracellularly, the type of cells with which the agent is contacted etc.

Inflammatory condition—a condition involving an inflammatory response which may result from a variety of causes such as a bacterial infection, a viral infection, over production of certain cytokines.

Autoimmune conditions—a condition involving activation and/or proliferation of immune cells, and/or anti-self auto antibodies, etc.

Transplantation rejection—a condition in which there is rejection in an individual of an organ or tissue transplant, such as, for example, bone marrow or stem cell transplants

Target cells—in metastasis, the cells with which the cancer cells react. In the process of moving through the blood or lymph the target cells are mainly intravascular and endothelial cells. In the process of homing and forming a new metastatic lesion, the target cells are cells or tissue in the organ or location where the cancer cells begin to proliferate to form the new lesion. In inflammation, the target cells are the cells or tissue with which the activated immune cells interact, typically, endothelial cells.

### BACKGROUND OF THE INVENTION

Metastasis is a main cause of death of cancer patients. The metastatic process begins by invasion of cancer cells from the primary tumor lesion into the blood or lymph vessels. The cancer cells move through the blood or lymph by a rolling process in which the cancer cells interact with intravascular and endothelial cells. During this process, the cancer cells extravasate from the blood or lymph vessels and form a new metastatic malignant lesion. The interaction between cancer cells and the endothelial cells is mediated by adhesion molecules expressed on the endothelial cells and on the cancer cells. The adhesion molecules on the endothelial cells that initiate their interaction with the cancer cells belong to a family of transmembrane molecules termed “selecting ” (Walz et al., 1990). The main type of selectin which has been shown to play an important role in the adhesion of cancer cells to endothelial cells is E-selectin (Kamagi, 1997). Metastatic cancer cells express selectin-ligands, two of the major ones being Sialyl Lewis-A (Sle^{a}) and Sialyl Lewis-X (Sle^{x}) (Kurahara, 1999).

For example, the level of expression of Sle^{a} or Sle^{x} on colon cancer cells has been shown to correlate with the risk of colon cancer patients to develop metastases (Ito et al., 1997). The level of expression of Sle^{a} but not Sle^{x}, was also correlated with incidence of metastasis in patients having oral squamous cell carcinoma (SCC).

The interaction between selectins expressed by endothelial cells and selectin ligands expressed by leukocytes is also a crucial factor during the inflammatory process in which the leukocytes must extravasate the blood vessels (Kong et al., 1993).

An essential component of the selectin ligands is GDP-L-fucose. The enzyme FX (GDP-keto-6-dioximannose-3, 5-epimerase, 4-reductase) is an intracellular enzyme responsible for the last step in the synthesis of GDP-L-fucose (Tonetti et al, 1996). FX catalyzes a combined epimerase and reductase reaction which converts GDP-4-keto-6-D-dioximannose to GDP-L-fucose. FX has been shown to. have a high homology with the murine protein P35B, which was, in itself, shown to be a tumor transplantation antigen (Szikora, J. P., et al., 1990).

GDP-L-fucose synthesized by FX is a substrate of several fucosyltransferases which are implicated in the biosynthesis of various lactoseamine glycoconjugates including blood groups, developmental antigens and also the selectin ligands SLe^{x} and SLe^{a}. The levels of mRNA of certain types of fucosyltransferases were shown to be increased in colorectal cancer tissues as compared to non-malignant colon tissue (Ito et al., supra). However, the increased expression of SLe^{a} in these cancer cells was not related to the content of the main type of fucosyltransferase enzyme involved in synthesis of SLe^{a} in the cells.

The E48 monoclonal antibody (MAb) recognizes an outer membrane antigen expressed by the majority of residual head and neck squamous carcinoma (HNSCC) cells (in humans) (Quak et al., 1990). E48 MAb was shown to eradicate small tumor deposits in HNSCC patients (De Bree, 1995) and an injection of radiolabeled E48 MAb to HNSCC-bearing nude mice resulted in complete remission of the tumors .(Gerretsen, 1994). The antigen to which the E48 MAb binds was characterized by cDNA cloning and found to be a glycosyl-phosphatidylinositol (GPI) anchored membrane protein expressed by squamous cells and having a high homology to the murine ThB protein which is a member of the Ly-6 gene family. Ly-6 is expressed on mouse lymphocytes and human keratinocytes, while the E48 antigen is expressed on keratinocytes.

### SUMMARY OF THE INVENTION

The present invention is based on findings which have shown for the first time that it is possible to regulate the level of activity of FX in cells by extracellular factors. Specifically, it was shown in accordance with the invention that the expression of FX can be upregulated in squamous cell carcinoma (SCC). cells as well as in activated leukocytes (particularly T-cells) by extracellular factors. Moreover, the cells in which FX was upregulated, were shown to bind, in vitro, to endothelial cells and to purified E-selectin while cells expressing low levels of FX did not bind to these cells or molecules. Moreover, it was shown that nude mice inoculated with cells expressing high levels of FX died more rapidly than nude mice inoculated with cells expressing lower levels of FX.

The effect on the level of FX in SCC cells was most prominent when the cells were contacted with specific antibodies. The level of FX in T-cells was regulated by specific combinations of antibodies and cytokines.

In some cells the upregulated expression of FX was correlated with increased expression levels of certain selectin ligands (SLe^{a} ligand in SCC cells and SLe^{x} ligand in activated T-cells).

The above findings of the present invention open the way for a new approach for the prevention and treatment of metastasis and of inflammation, auto-immunity and transplant rejection.

In accordance with a first aspect of the invention, the invention thus provides a method for inhibiting or preventing adherence of cells to their target cells or target tissue comprising reducing the level of activity of FX in said cells.

In accordance with one embodiment, the cells are cancer cells and their target tissue is endothelium and the tissue in the organ into which they metastasize.

In accordance with an additional embodiment, the cells are leukocytes and their target tissue is endothelium or other leukocytes.

The first aspect of the invention concerns methods and agents for down-regulating or up-regulating the level of activity of FX in cancer cells. In accordance with this aspect of the invention, a method is provided for inhibiting or preventing metastasis of cancer cells comprising reducing the level of activity of FX in cancer cells.

In accordance with one embodiment, the reduction in the level of activity of FX in the cancer cells results from contact of the cancer cells with an agent which reduces the level of FX in said cells. Thus, a method is provided for inhibiting or preventing metastasis of cancer cells comprising contacting said cells with an effective amount of an agent which reduces the level of activity of FX in said cancer cells.

In accordance with the invention, an agent may be selected based on its ability, upon contact with the cancer cells, to reduce the activity. of FX as this is defined above.

In accordance with one embodiment, the agent in accordance with the invention, reduces the activity of FX without affecting the amount of the protein. The agent may in itself be a competitor substrate, an inhibitor of one of the two enzymatic activities of FX, epimerase or reductase, or one which effects the level of expression of the protein either on the transcription level or on the translation level of the protein. Alternatively, the agent may reduce the activity of FX by reducing its amount such as, for example, by causing its degradation or by inhibiting the transcription or translation of its coding sequences.

In some cases, the reduction in activity of FX may result in reduced expression of selectin ligands on the cancer cells as compared to the expression of these ligands on the cells prior to reduction of the FX activity. However, in accordance with the invention, the inhibition or prevention of metastasis by reducing the activity of FX in the cells may, in some cases be obtained via pathways which do not involve altered expression of selectin ligands.

In accordance with one embodiment, the agent of the invention is an extracellular agent.

Such an extracellular agent may, for example, be an antibody which binds to an extracellular moiety on said cells or fractions or derivatives of such an antibody (e.g. Fab, Fc, single chain antibodies, etc.), which essentially maintain the antibody's binding characteristics. A specific example of such an antibody is the anti E48 ab. The agent may also be a proteinaceous, carbohydrate or lipid molecule capable of binding to membrane receptors or capable of penetrating the membrane into the cell.

In accordance with an additional aspect of the invention, an agent is provided which enhances the level of activity of FX in cancer cells. Such enhancement of FX expression may be useful, for example, to increase immunogenecity of cancer cells included in a cancer vaccine. The agent may, for example, be an antibody which binds to a membrane receptor on the cancer cells resulting in up-regulation of the level of activity of FX in the cells. A specific example of such an antibody is the anti E48 antibody mentioned above and below. Typically, in accordance with this aspect, cancer cells intended to be included in a vaccine will first be incubated with the agent which enhances the level of activity of FX in the cells for an appropriate period of time. Following enhancement of the level of the FX enzyme, the cells will then undergo additional treatments for their reparation as a vaccine (such as, for example, irradiation which will prevent further proliferation of the cells). Typically, in accordance with this embodiment, the enhancement of the level of activity of FX in the cells will be an enhanced level of expression of the FX protein in the cells. In addition, a vaccine comprising an effective amount of cancer cells expressing a high level of activity of FX for use in the prevention or treatment of cancer is provided. In this case, the effective amount of the active agent is an amount which, upon contact with the cells, will result in a substantive increase in the level of activity of FX in the cells. In addition, a method is provided for enhancing the immunogenicity of cancer cells comprising contacting said cells with an effective amount of an agent which enhances the level of activity of FX in said cells.

An additional aspect of the invention concerns down-regulation and up-regulation of the activity of FX in leukocytes.

In accordance with one aspect of the invention, the level of activity of FX in leukocytes, particularly in T-cells is down-regulated to inhibit or prevent the activation of the leukocytes and their extravasation from blood or lymph vessels in conditions of undesired inflammatory processes, auto-immunity and in transplant rejection. In accordance with the findings of the present invention it has been shown that often, activation of immunocytes, particularly T-cells, by various factors results in elevation of the activity of FX in the cells as this term is defined above. Thus in accordance with the invention, a method is provided for the inhibition or prevention of an undesired inflammatory response, an auto-immune process or transplant rejection comprising reducing the level of activity of FX in said leukocytes.

By a preferred embodiment, the leukocytes are T-cells, most preferred CD4⁺ T-cells.

In accordance with an additional embodiment of this aspect of the invention. a method is provided for inhibition or prevention of an inflammatory response, an auto-immune process or transplant rejection comprising contacting leukocytes of a treated individual with an effective amount of an agent which inhibits or prevents the activity of FX in said leukocytes. Such an agent may, for example be an antagonist or analog of a cell growth factor which was altered in a manner which enables it to bind to the cells without resulting in elevation of the level of FX in the cells. Such an agent may block the activity of another activating agent and prevent it from elevating the level of FX in the cells.

In accordance with one embodiment, the reduction in activity of FX results from inhibition of activation of FX by an activation factor in said leukocytes. By a preferred embodiment, said leukocytes are T-cells.

In accordance with an additional aspect of the invention, the activity of FX in leukocytes, particularly in T-cells is enhanced. This may be useful for enhancing a desired immune reaction such as for example, in cases of reduced immunity of an individual such as in an individual suffering from genetic or acquired immune deficiency. Thus, a method is provided for enhancing a desired immune reaction comprising elevating the level of activity of FX in leukocytes involved in said reaction.

The invention further provides a diagnostic aspect in accordance with which the level of FX in cancer cells is used as a diagnostic and prognostic factor. The level of FX in cancer cells may be used for staging the disease as well as for indicating the potential of the cancer cells to form metastasis. Thus, a method is provided for determining the stage of a malignant disease involving cancer cells comprising analyzing the level of activity of FX in said cancer cells, comparing said level to the level of activity of FX in cancer cells being in different stages of the disease to find a level of activity of FX essentially equal to the level of FX expression in the tested cells and determining the stage of the malignant disease.

In addition, a method is provided for determining the probability of formation of metastasis by said cancer cells comprising measuring the level of activity of FX in said cancer cells, comparing said level to the level of FX in non-malignant cells, a measured level higher than that of the level in non-malignant cells indicating a high probability that said cells will form metastasis. The comparison of the measured level of FX may also be to a predetermined threshold level which is calculated on the basis of a number of measurements in non-malignant cells, a measured level higher than the threshold level indicating a high probability that said cells will form metastasis.

In accordance with an additional diagnostic aspect of the invention, the level of FX in immunocytes (particularly in T-cells) may be used as a marker of activation which may, for example, be useful in determining an individual's response to an immunogen. Thus the invention provides a method for determining an individual's immune response to an immunogen comprising administering said immunogen to the individual; determining the level of activity of FX in immunocytes of said individual; comparing the level of activity of FX in said cells to the level of activity of FX in immunocytes of a non-immunized individual who was not administered with the immunogen, a level of measured FX activity higher than the level of FX activity in immunocytes of said non-immunized individual indicating a high probability of an immune response in said individual. As described above, the measured level of FX may be compared to a predetermined threshold level calculated on the basis of measurement of activity of FX in leukocytes of at least two non-immunized individuals to whom the immunogen was not administered.

### DETAILED DESCRIPTION OF THE INVENTION

### EXAMPLES

The various aspects of the invention will now be illustrated by the following non-limiting Examples with occasional reference to the attached Figures.

### BRIEF DESCRIPTION OF THE FIGURES

FIG. 1 is a graphic representation obtained by FACS analysis showing the expression of E48 on HNSCC. The horizontal line in each graph represents the cell population gated for E48 expression.

FIG. 2 is a photograph showing FX mRNA expression in Northern blots prepared from control (−Ab) and αE48 MAb-treated (+Ab) 22A-WT cells. A northern blot of rRNA prepared from the same cells is shown as control. The northern blot shown is a representative of eight experiments.

FIG. 3 is a photograph showing FX protein expression in Western blots prepared from 22A-WT cells treated with αE48 MAb. The cells were incubated with the antibody for 60, 120 and 189 minutes. Cell lysates were analyzed by immunoblot using rabbit anti FX antibodies. Maximal FX up-regulation occurred after 120 min. incubation. Actin expression in the tested cells was shown as control.

FIG. 4 shows a photograph of northern blots prepared from 22A-WT cells incubated with antibodies directed against CD59 (αCD59), NCA (αNCA) and ICAM (αICAM) (FIGS. 3C, D and E respectively) which did not upregulate FX mRNA expression and of 22A-WT cells treated with αE48 MAb (α48) (FIG. 3B) which showed upregulated levels of FX 22A-WT cells incubated in growth medium were used as negative controls (FIG. 3A). Northern blots showing rRNA expression in all the above cells are shown as control. The northern blot shown is a representative of three experiments.

FIG. 5 is a photograph showing FX mRNA expression in northern blots prepared from HNSCC expressing either high or low levels of E48. A northern blot of rRNA prepared from the same cells is shown as control. The northern blot shown is a representative of three experiments.

FIG. 6 is a photograph showing FX protein expression in Western blots prepared from by HNSCC expressing either high or low levels of E48. A western blot showing actin expression in the same cells is shown as control. The western blot shown is a representative of three experiments.

FIG. 7 is a graphic representation showing flow cytometry data of Sialyl-Lewis-a and VIM2 expressed in HNSCC variants expressing high levels of E48 and in variants expressing low levels of E48. The flow cytometry data shown represent the results of five experiments. %P=% positive cells. M=mean fluorescence.

FIG. 8 is a graphic representation showing the expression of Sialyl-Lewis-a in αE48-MAb-treated HNSCC (E48) and in control cells which were either treated with αNCA MAbs (+NCA) or were untreated (−Ab). The values represent % of Sialyl-Lewis-a expressing cells as determined by flow cytometry. The % of SLe^{a} positive 22A E48^{lo} cells was significantly (P<0.005) higher among αE48 MAb treated cells than among control cells. The % of SLe^{a} positive cells increased also in the MAb-treated 22A-WT population as compared to controls, but the difference was not statistically significant. The data shown represent the mean of three experiments.

FIG. 9 is a graphic representation showing rolling of E48^{lo} 14C and E₄₈^{hi} 14C cells [14C-CMV16] on purified E-selectin and on activated endothelial cells under physiological shear flow.

FIG. 9(A) shows accumulation of E48^{lo} (L) and E48^{hi} (H) cells on a plastic plate coated with an E-selectin-IgG chimera (E-selectin) adsorbed onto protein A substrate and assembled on the lower wall of a parallel plate flow chamber. Cells (10⁶/ml) were perfused at room temperature through the chamber at a shear stress of 1 dyn/cm² in binding medium alone or in the presence of 5 mM EDTA (Erg+EDTA). The number of cells which accumulated and maintained rolling at the end of 1 min of perfusion in two microscopic fields was determined as described in materials and methods. No cells tethered to control substrates coated with protein A alone. After 1 min perfusion, adherent cells were subjected to an abrupt 5-fold increase of shear stress and the number of cells that remained bound for at least 5 sec after the increase of shear was determined. The mean number +/− range of cells accumulated at 1 dyn/cm² and of accumulated cells remaining stably bound at 5 dyn/cm² on two fields of view are shown for the E48^{lo} and E48^{hi} cells. All adherent E48^{hi} cells maintained persistent rolling on the selectin-coated substrate. Mean rolling velocities of cells±S.E.M. at 1 and 5 dyn/cm² were 4.5+/−0.5 micron/sec and 5.3+/−0.6 micron/sec, respectively. Data are representative of four independent experiments.

FIG. 9(B) shows the number of rolling events of E48^{lo} and E48^{hi} 14C cells perfused over identical monolayers of TNFα-stimulated HUVEC. Cells were perfused as described in part A, but adherent E48^{hi} cells rolled faster on the HUVEC than on purified E-selectin (mean velocity of 10.3 micron/sec), resulting in their low accumulation. The total number of rolling events i.e. tethers followed by persistent rolling of at least 3 sec was therefore determined. In the blocking experiments, activated HUVEC were pretreated for 20 min with saturating levels of the E-selectin blocking MAb [1.2B6 (Serotech, Oxford, UK)] before cell perfusion. Pretreatment of TNFα-activated HUVEC with murine IgG control had no effect on the number of E48 transfected cells tethered and rolling relative to untreated TNFα-activated HUVEC (not shown). The results shown are the mean of data determined in two experimental fields and they represent one of three independent experiments.

FIG. 10 is a graphic representation showing the level of expression of FX mRNA (as measured in Northern blots) as compared to the level of Sle^{a} expression (as measured by FACS) in cells originating from nine different colon cancer cell lines.

FIG. 11 is a photograph showing FX mRNA expression in Northern blots prepared from non activated peripheral blood lymphocytes (PBL), PBLs activated with PHA and PBL activated with PHA and IL-2 for a period of 48 and 72 hours.

FIG. 12 is a photograph showing FX mRNA expression in Northern blots prepared from non activated PBLs or PBLs activated with PHA+IL-2 for 24 hours, 48 hours or 96 hours. As control, Northern blots prepared from ribozymal RNA of the cells are shown.

FIG. 13 is a photograph showing FX mRNA expression in Northern blots prepared from CD4⁺ cells incubated in growth medium, with anti-CD3 or with anti-CD3 and anti-CD28. Northern blot cells ribozymal RNA of the cells are shown as control.

### I. EXPERIMENTAL PROCEDURES

Cell Lines and Tissue Culture

The HNSCC (Krause, C., et al., Arch. Otolaryngol 107:703, 1981)) lines UM-SCC-22A (22A-WT) and UM-SCC-14C (14C) were kindly provided by Dr. T. E. Carey (Ann Arbor, Mont., U.S.A.). 22A WT cells highly express the E48 antigen, whereas E48 was not expressed on 14C cells. We also studied 22A-WT cells selected by flow cytometry sorting for a high expression of E48 (22A-E48^{hi} ), 22A-WT cells transfected with E48 antisense (clone 8-3) which expressed low levels of E48 (E48^{lo}) and 14C cells transfected with E48 cDNA (14C-CMV16 (E48^{hi})).

Cells were routinely cultured in humidified air with 5% CO₂ at 37° C. in DMEM (Biological Industries, Beit-Ha'Emek, Israel), 5% FCS (Hyclone, Logan, Utah, U.S.A.), 2 mM L-glutamine, 1% penicillin/streptomycin (Biological Industries, Beit-Ha'Emek, Israel).

Human umbilical cord vein endothelial cells (HUVEC) were isolated from umbilical cord veins according to the method of Jaffe et al. (1973), pooled and established as primary cultures in M199 containing 10% FCS, 8% pooled human serum, 50 μg/ml endothelial cell growth factor (Sigma Israel Chemicals Ltd., Rehovot, Israel), porcine intestinal heparin (10 U/ml) (Sigma Israel Chemicals Ltd., Rehovot, Israel) and antibiotics. Primary cultures were serially passaged (1:3 split ratio) and passages 3-4 were taken for adhesion experiments.

Antibodies

Mouse monoclonal antibody (MAb) against E48 has been described previously (Quak et al., ). Mouse MAb against human VIM-2 (αVIM-2) (Kniep, et. al. 1996), was kindly supplied by Dr. V. Knapp, Institute of Immunology, Vienna University, Austria. Mouse MAb against human SLe^{a} (αSLe^{a} clone 203) has been described previously (Takada, et al., 1991). Mouse MAb against human SLe^{x} (SLe^{x}—clone Km-93) was purchased from Serotech, Oxford, UK). Mouse MAb against human NCA (αNCA) was kindly supplied by Dr. D. Goldenberg and Dr. H. Hansen, Immunomedics, Inc., New Jersey, USA. This antibody is also crossreactive with the GPI linked 50/90 antigen which is expressed on active granulocyte. Mouse MAb against human CD59 (clone YTH53.1) and ICAM-1 (clone 84H10) and against human CD62E (E-selectin—clone 1.2B6) were purchased from Serotech, Oxford, UK. A rabbit MAb against human actin was purchased from Sigma (St. Louis, Mo., U.S.A.). Polyclonal rabbit antibodies directed against FX were generated in our laboratory, using two peptides from the native human FX protein:

(1) H2N-CNGPPMNSNFGYS (aa133-144) and

(2) H2N-CASNSKLRTYLPDFRF (aa284-298).

Generation of Sense and Anti-Sense E48-cDNA Transfected HNSCC Cell Lines

The E48 encoding cDNA in pCDM8 (Brakenhoff, et al., Immunol., 159:4879-4886, (1995)) was inverted to antisense orientation by EcoRI cleavage and religation. The isolated inserts of the sense and antisense cDNAs were excised by HindIII/NotI digestion and inserted in the HindIII/NotI sites of pRC-CMV (Invitrogen, Leek, The Netherlands). The cell lines were transfected by lipofectin (GibcoLife Technologies, Breda, The Netherlands). In short, cells were plated in a 6 well plate (2×10⁵ cells/well) and cultured overnight. A mixture of lipofectin and DNA was prepared: 10 μg/ml lipofectin and 10 μg DNA for UM-SCC-14C or 50 μg/ml lipofectin and 10 μg DNA for UM-SCC-22A. The lipofectin and DNA were mixed in a small volume of serum-free DMEM (20% of the final transfection volume) and incubated at room temperature for 15 minutes. The solution was adjusted to the final volume with serum-free DMEM. The cultured cells were washed twice with serum-free medium before the addition of the transfection solution (0.75 ml/well). After 24 hrs incubation, the transfection solution was replaced by regular tissue culture medium. After an incubation period of 72 hrs selection medium was added containing 1 mg/ml G418 (GibcoLife Technologies, Breda, The Netherlands). Surviving clones were tested for E48 expression by immunocytochemical staining. From transfectants with a heterogenous expression, or a down-regulated expression of E48, clones with a homogeneous E48 expression were obtained by limiting dilution.

Sorting of 22A-WT Cells for High E48 Expression

3×10⁶ cells were incubated for 60 min at 4° C. with anti E48 MAb. Following two washes with DMEM supplemented with 5% FCS, the cells were incubated with a FITC-conjugated secondary mouse antibody against human IgG. After two more washes, cellular populations expressing high levels of E48 (22A E48^{hi}) were sorted using a FACS-IV sorter (Becton Dickinson, Mountain View, Calif., U.S.A.).

E48-Mediated Signal Transduction and Differential Gene Expression Analysis

E48-mediated signals were transduced to HNSCC by incubating 22A-E48^{hi} cells with E48 for 1 hour at 37° C. Control cells were incubated under the same conditions without antibody. The cells were subsequently washed and RNA isolated. Differentially expressed genes were determined by differential display polymerase chain reaction (DD-PCR) (Liang, et al, Science, 257:967-971 (1992)) using the Delta™ RNA Fingerprinting kit (Clontech Laboratories, Inc., California, USA). The following primers were used:

5′-CATTATGCTGAGTGATATCTCTTTTTTTTTGC-3′ and
5′-ATTAACCCTCACTAAATGGAGCTGG-3′.

Differentially expressed cDNA bands in antibody-stimulated cells as compared to control cells, were eluted from the gel and re-amplified by PCR using the same primers according to the manufacturer's instructions. A higher expression of mRNA corresponding to the cDNA identified in the above assays was confirmed by using Northern blotting of RNA from untreated or from αE48-treated cells. Differentially expressed cDNA bands were isolated from the sequencing gel, radiolabeled, and used as a probe in RNA blot analysis. The corresponding cDNA fragment that generated a specific hybridization pattern on RNA blots was sequenced in both directions, and the nucleotide sequence obtained was compared with known sequences by searching the GenBank with the FASTA program (Genetic Computer Group software (Madison, Wis., U.S.A.).

RNA Isolation and Northern Blotting

Total RNA was isolated from antibody-treated or control cells using RNAzol solution (Bio Labs, Jerusalem, Israel). A total of 20 μg of RNA was loaded on an 1% agarose formaldehyde gel and electrophoresed in MOPS buffer as described by Sambrook et al., Molecular Clong: a Laboratory Manual, Cold Spring Harbor Laboratory Press, (1989). The RNA was subjected to Northern blotting by capillary transfer in 20×SSC onto hybond N membrane (Amersham, Aylesbury, UK), and hybridized as described below.

Probes

Differentially expressed cDNA bands were excised from the DD sequencing gel and labeled with [α-³²P]dCTP (3000 Ci/mmol) (Hybond™-N, Amersham Int., Buckinghamshire, UK), by multiprime elongation (Boehringer Mannheim, Mannheim, Germany) and hybridized overnight at 42° C. to filters. The filters were washed once in 2×SSC/0.1% SDS at room temp. for 30 min followed by two washes in 0.1% SSC/0.1% SDS for 30 min each at 50° C., and autoradiographed for one to three days.

Flow Cytometry

1×10⁶ cells were incubated for 60 min at 4° C. with the different MAbs (diluted 1:80 to 1:100). Following two washes with DMEM supplemented with 5% FCS and 0.05% sodium azide, the cells were incubated with FITC-conjugated secondary antibodies (goat anti mouse IgG, or goat anti mouse IgG Fab′ (Jackson ImmunoResearch Lab. Inc., West Grove, Pa., U.S.A.). Following two more washes, the pattern of antigen expression was determined using a Becton Dickinson FACSort (Mountain View, Calif., U.S.A.) and the CellQuest software.

Sialyl Lewis-a Expression Following E48 Ligation

2×10⁶ 22A WT cells were seeded in 25 cm culture flasks in 5 ml DMEM supplemented with 5% FCS. After 24 hrs the medium was replaced with 2 ml fresh medium with or without 50 μg/m monoclonal antibody against E48 or NCA for 2, 4, 6 or 8 hours At the end of incubation, the cells were removed from the bottom of the flask and SLe^{a} expression was determined by flow cytometry using as first antibody a 1:10 diluted biotin-conjugated IgM mouse antibody directed against human SLe^{a} (Seikagaku Co., Tokyo, Japan) and as secondary Ig antibody Phycoerythrin-conjugated Streptavidin (Jackson ImmunoResearch Lab. Inc., West Grove, Pa., U.S.A.).

Statistical Evaluation

Student's t-test was used to evaluate the statistical significance of differences between SLe^{a} expression on HNSCC before and after treating the cells with αE48 and αNCA antibodies.

SDS-PAGE and Immunoblotting

E₄₈^{hi} or E48^{lo} HNSCC were cultured in monolayer to confluence and lysed on their culture dishes with Laemmli sample buffer (Laemmli, 1970). Lysates were boiled for 10 min, centrifuged and applied on a miniprotean II system (BioRad Labs, Hercules, Calif., U.S.A.) for SDS-PAGE using a 12% slab gel as described by Laemmli. Electrophoretic transfer of proteins from polyacrylamide gel to nitrocellulose (Schleicher and Schull, Dassel, Germany) was performed by a mini-transblot electrophoretic cell (Bio-Rad, Hercules, Calif., U.S.A.) at 100V for 1.5 hours. After transfer, the nitrocellulose membrane was cut into strips and incubated at room temp. with 5% milk in TBS-tween for 30 min to block free binding sites on the membrane.

The blocked nitrocellulose membrane strips were incubated overnight with anti FX polyclonal antibodies raised in our laboratory, diluted 1:2000, and then washed 3×10 min with Tween-20 buffer, and incubated for 45 min with HRP-conjugated secondary goat antibody against anti rabbit IgG at room temp. Finally, the nitrocellulose strips were washed 3×10 min with TBS-tween. The bands were visualized by chemoluminescence-ECL reaction and autoradiography by exposure to Kodak X-AR5 film (Eastman Kodak Co., Rochester, N.Y., U.S.A.) for 1-8 min.

The total amount of protein in the lanes was verified by immunoblotting the membrane with anti-actin antibodies diluted 1:2000.

Laminar Flow Assays

Cultured IUC cells grown as monolayers were harvested by a 10 min incubation with H/H medium (Hanks Balanced Salt Solution, HBSS (Sigma Israel Chemicals Ltd., Rehovot, Israel), containing BSA (2 mg/ml, fraction V, Sigma) and 10 mM HEPES, pH 7.4), supplemented with 5 mM EDTA at 37 Washed cells were resuspended in the same medium at a concentration of 1×10⁷ cells/ml and kept at room temperature until use. Cells were diluted 20 fold into binding medium (H/H supplemented with 2 mM Ca²⁺) and immediately perfused through the flow chamber. An E-selectin coated substrate was prepared as described (Fuhlbrigge, et al., J. Cell. Biol., 135:837-848, (1996)). Briefly, protein A (20 μg/ml in coating medium; Sigma) was spotted onto a polystyrene plate, the substrate was blocked with 2% human serum albumin (HSA, Fraction V (Calbiochem, La Jolla, Calif., U.S.A.)) in PBS and overlaid with culture supernatant from COS cells transfected with cDNA of human E-selectin-IgG1 (a kind gift of Dr. T. S. Kupper, Brigham and Women's Hospital, Boston, Mass., U.S.A.). For control, a protein A spot was overlaid with culture supernatant from untransfected COS cells. The E-selectin coated plate or the protein-A control plate were assembled in a parallel flow chamber (260 μm gap thickness) (Lawrence et al., Cell, 65: 859-873, (1991)) and mounted on the stage of an inverted phase-contrast microscope (Diaphot-TMD, Nikon Inc., Garden City, N.Y.). For adhesion experiments on resting or activated EC, primary HUVEC (passage 2 or 3) were plated at confluent density for 1 hr on tissue culture dishes (Becton Dickinson, Falcon Plates, Plymouth, UK) spotted with human fibronectin (25 μg/ml in PBS). Nonadherent EC was gently rinsed out and adherent cells were grown on the fibronectin-coated spots for 24 hrs before cytokine treatment. The EC monolayers were left intact or stimulated for 18 hrs with heparin-free culture media supplemented with TNFα (2 ng/ml, 50 units/ml) (R&D, Minneapolis, Minn., U.S.A.). Before assay, the various EC-coated plates were washed three times with binding medium and assembled as the lower wall of the flow chamber, where a portion of the monolayer (5×30 mm) was exposed to flow. 5×10⁵/ml cells suspended in binding medium were perfused in the flow chamber with a syringe pump (Harvard Apparatus, Natick, Mass.) attached to the outlet side. Cells were visualized with a 10× objective and videotaped with a long integration LIS-700 CCD video camera (Applitech, Holon, Israel) and a Time Lapse SVHS-Video recorder (AG-6730, Panasonic, Japan). The number of cells that accumulated in two representative fields (each 0.17 mm² in area) during 1 min of constant flow generating a wall shear stress of 1 dyn/cm² was manually quantitated by analysis of played back images directly from a monitor screen. For inhibition studies, substrates were washed with H/H medium supplemented with 5 mM EDTA. The cells were then suspended in the same medium and perfused through the chamber in a wall shear stress of 1 dyn/cm². Rolling velocities were measured for cells accumulated on the E-selectin substrate during 1 min of flow at 1 dyn/cm². Rolling of cells accumulated at low flow and then subjected to elevated shear stresses of 5, 10 and 15 dyn/cm², each shear increment lasting for 10 seconds was determined thereafter.

Isolation of Normal Human PLB

Cells were isolated by standard protocols from leukocytes obtained from healthy volunteers. PBMC were isolated by Ficoll-Hypaque centrifugation and recovery of cells at the interface.

Injection of Cells Expressing High Levels of E-48 and Cells Expressing Low Levels of E-48 to Nude Mice

22A-WT cells expressing high levels of E-48 (sorted by FACS as explained above) and cells expressing low levels of E-48 (comprising anti-sense E48-cDNA as explained above) were injected into nude mice. 10⁶ cells of each of the above kinds of cells were injected subcutaneously to the neck of the animals. The mortality rate of the mice was determined by counting the live mice in each age every several days.

### II. RESULTS

1. Ligation of E48 by αE48 MAb Upregulates the Expression of FX

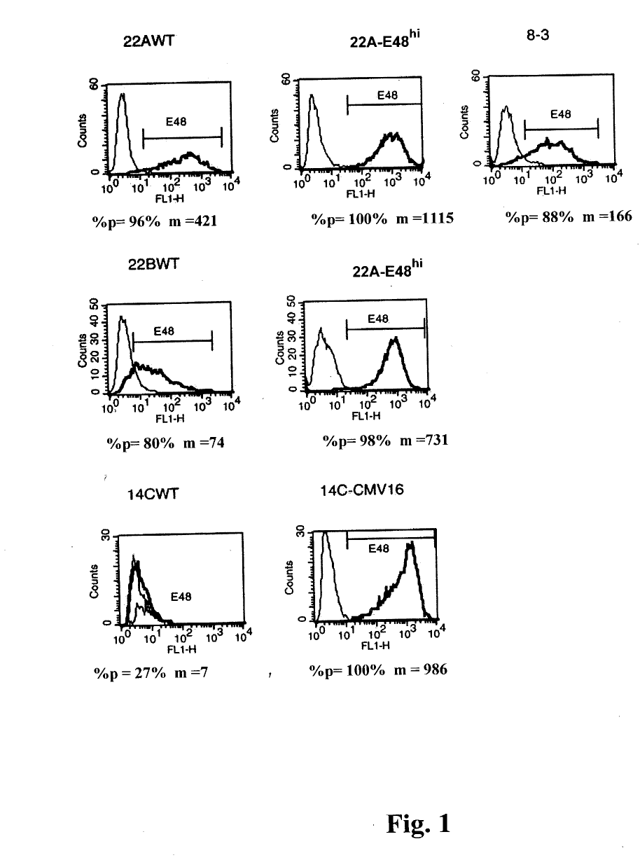

The level of expression of E48 on various nonstimulated HNSCC lines used in the study was determined by flow cytometry as explained above and seen in FIG. 1.

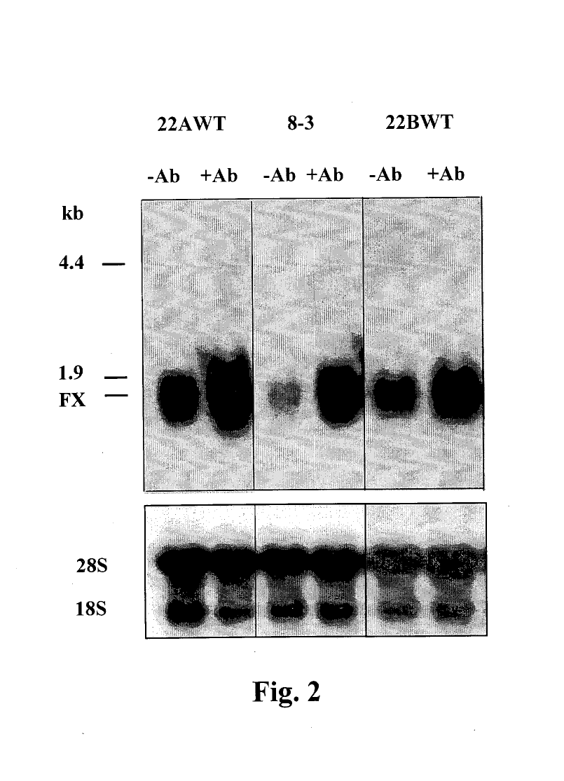

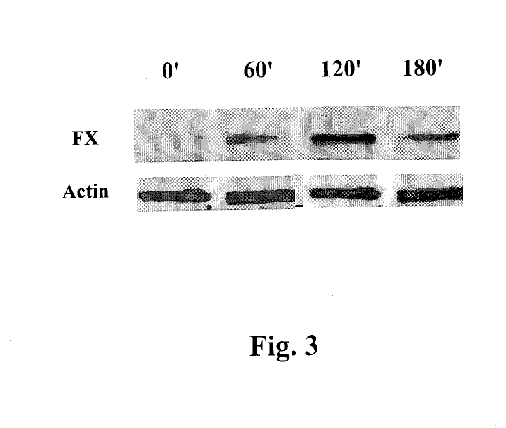

αE48 MAb was added to 22A-WT cells as a surrogate ligand as the physiological ligand of E48 has not been identified thus far. The method used to detect altered gene expression was Differential Display PCR (DD PCR) of mRNA. 22A-WT cells treated with αE48 MAb for 60 min at 37° C. were used in the DD assay. 22A-WT cells incubated under the same conditions but without antibody served as controls. The density of several cDNA bands was increased in antibody-stimulated cells. Multiple repetitive experiments yielded the same results. One of these cDNA bands was then eluted from the gel and amplified. A higher expression of a rRNA species corresponding to the cDNA species identified in the above assays was confirmed using Northern blotting of RNA from untreated or αE48 MAb-treated 22A-WT cells. The corresponding cDNA was then cloned and sequenced. Gene-bank analysis showed that this cDNA species had a 98.2% homology to FX [(Tonetti supra, 1996), accession no. U58766)]. FIG. 2 shows that compared to control cells FX mRNA is upregulated in 22A-WT cells by αE48 MAb ligation. Similar results were obtained with 22A-WT cells incubated with αE48 MAb conjugated to polystyrene 6μ microparticles (Polysciences Inc., Huntsville, Ala., U.S.A.). An exposure of cells to E48 MAb-conjugated beads for 30 min yielded a maximal up-regulation of FX (results not shown). The up-regulation of FX in 22A-WT cells by E48 ligation was also demonstrated at the protein level. Western blotting of lysates from αE48 MAb treated cells as well as from control cells showed an increased expression of FX protein in antibody-treated cells. Two hours of incubation yielded a maximal up-regulation of FX (FIG. 2).

2. FX Up-Regulation by E48-Mediated Signaling

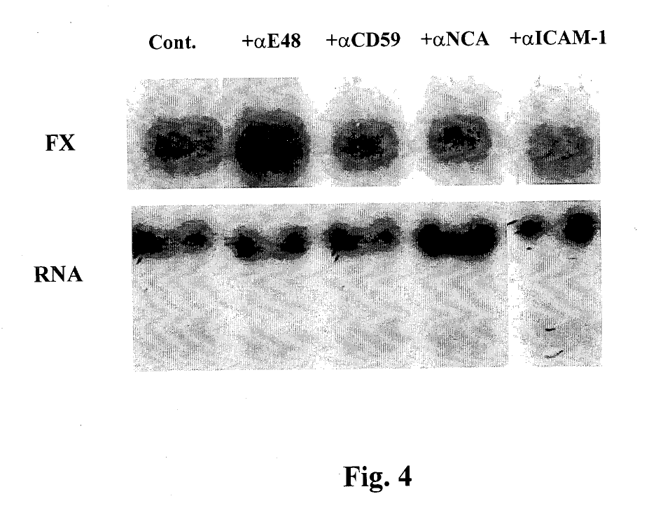

It was first established that 2 GPI-linked proteins, CD59 (Sugita, Y. et al, Immunotechnology, 3:157-168, (1995)) and NCA (Hansen, H. J., et al., Cancer, 71:3478-3485, (1993)) are expressed by the HNSCC lines used in this study. As seen in FIG. 4, exposure of 22A-WT cells to MAb directed against CD59 and NCA, did not result in FX mRNA up-regulation while the same cells exposed to αE48 MAb under identical conditions yielded the expected FX up-regulation.

As also seen in the figure, incubation of 22A-WT cells with antibodies directed against ICAM-1, a non-GPI-anchored protein expressed on these cells, did not upregulate FX mRNA expression in these cells, as compared to up-regulation seen in the cells incubated with αE48 MAb (serving as a positive control in these experiments).

3. The Expression of FX was Downregulated in an E48 Anti-Sense Transfectant and Upregulated in an E48 cDNA Transfected HNSCC

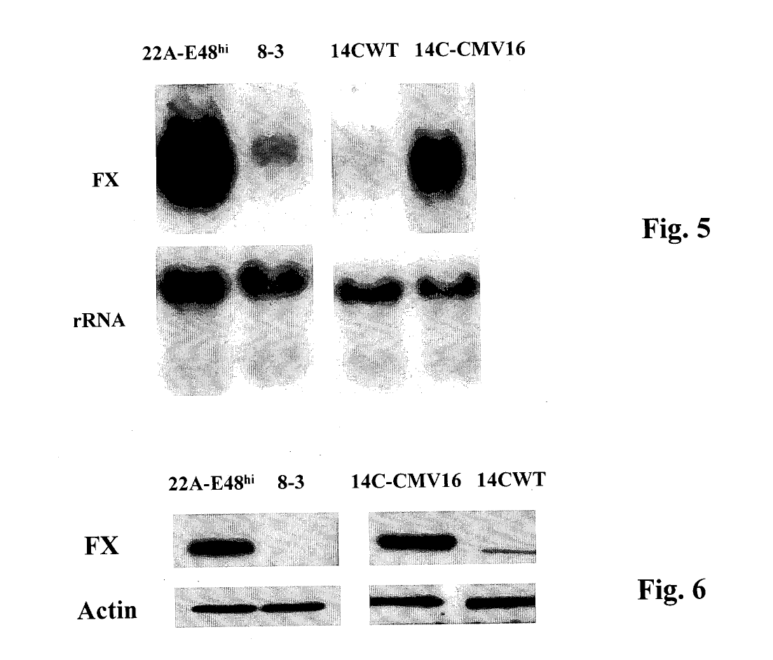

Northern blotting showed a positive correlation between E48 and FX expression in the two sets of E48^{hi}/E48^{lo} HNSCC lines tested (FIG. 5). Moreover, when the E48^{lo} 14C line was transfected with E48 cDNA and as a result expressed high levels of the E48 protein, a concomitant significant increase in the expression levels of FX was seen in the transfectants as compared to the untransfected controls (FIG. 5).

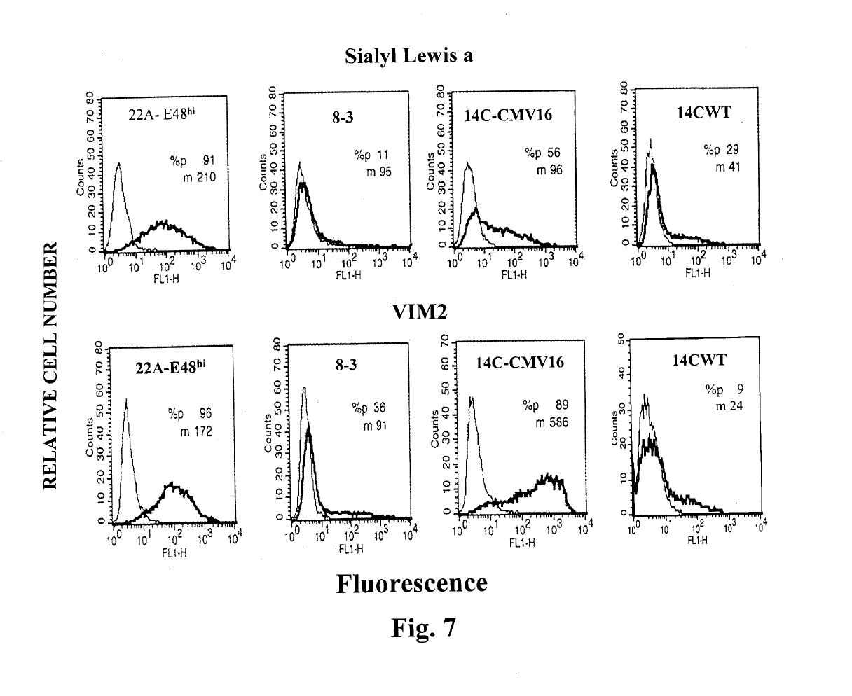

A positive correlation between E48 and FX levels in HNSCC was also demonstrated at the protein level (FIG. 6). As seen in the figure, Western blots of lysates from the two sets of E48^{hi} and E48^{lo} cells used in this study. E48^{hi} cells expressed significantly higher levels of FX protein than E48^{lo} cells.

4. The Expression of Fucosylated Glycans on HNSCC Increases Following E48 cDNA Transfection and Decreases Following E48 Antisense Transfection

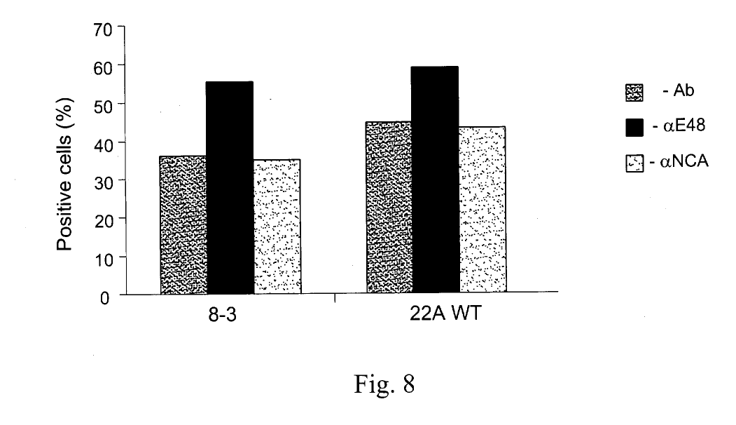

In view of the fact that GDP-L-fucose is the key substrate of several fucosyl transferases implicated in the biosynthesis of diverse lactosamine glycoconjugates including the selectin ligands SLe^{x} and SLe^{a} the expression of SLe^{a} and SLe^{x}, by the two sets of HNSCC expressing either high or low levels of E48 and FX was compared by flow cytometry. The results shown in FIG. 7 show that E48/FX^{hi} HNSCC express significantly higher levels of SLe^{a} than E48/FX^{lo} cells. Neither E48/FX^{hi} nor E48/FX^{lo} HNSCC expressed SLe^{x} (data not shown), consistent with the lack of Sle^{x} expression in many tumor cells. The expression of VIM-2, another major fucosyl sialo-lactosamine not recognized by selectins, was assayed next. It was found that E48/FX^{hi} cells expressed significantly higher levels of VIM-2 than E48/FX^{lo} cells (FIG. 7). This shows that a higher level of distinct fucosylated glycans is produced by E48/FX^{hi} cells than by E48/FX^{lo} cells.

5. Ligation of E48 Upregulates the Expression of SLe^{a}.

The next set of experiments was performed in order to directly test whether E48 ligation controls the level of SLe^{a} biosynthesis through a cascade of events initiated by E48-mediated signaling through up-regulation of FX and resulting in an upregulated expression of certain E-selectin ligands. 22A-WT and 8-3 (E48^{lo}) cells were incubated with αE48 MAb for 2, 4, 6 and 8 hours. Expression of SLe^{a} by these cells and by 22A-WT and 8-3 (E48^{lo}) control cells incubated either with an αNCA MAb (see above) or in medium) was then assayed by flow cytometry utilizing strepavidin-labeled anti SLe^{a} antibodies.

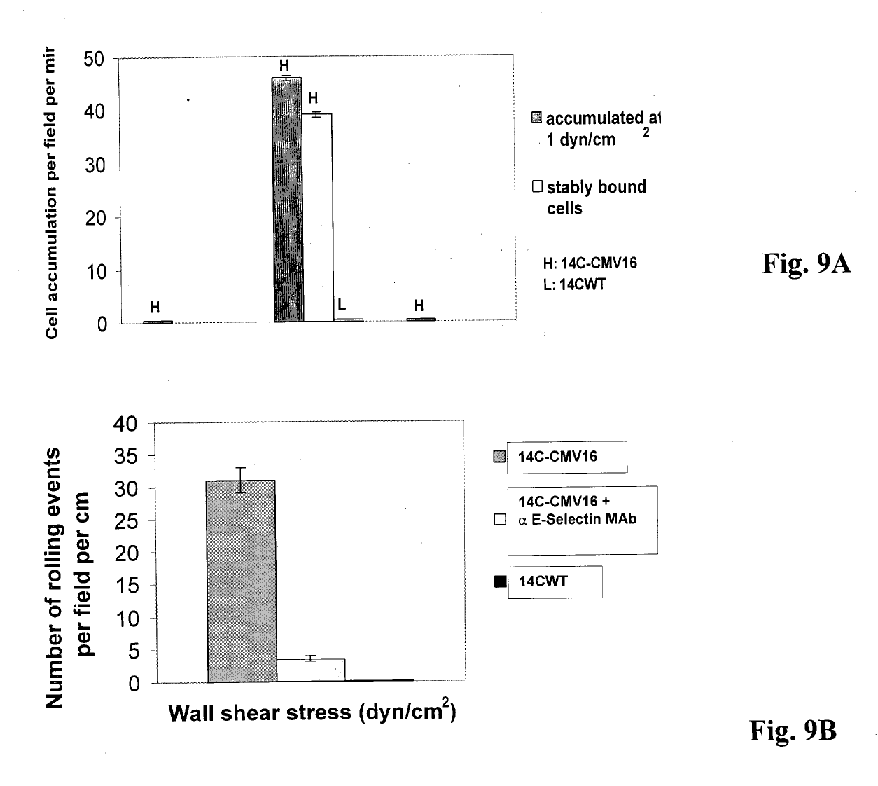

As seen in FIG. 8 an upregulated expression of SLe^{a} occurred in the αE48 MAb-treated cells compared to control ones. However, the up-regulation was more pronounced in the 8-3 E48^{lo} cells which expressed a low basal level of SLe^{a} (FIG. 6). In these cells the increase in SLe^{a} expression was significant (P<0.005). The upregulated expression of SLe^{a} following αE48 ligation was seen already after two hours of incubation with the αE48 MAb, with the highest up-regulation achieved after six hours of incubation (results not shown).

An E48-ligation mediated small increment in the expression levels of SLe^{a} on 22A-WT cells was repeatedly obtained, although these cells expressed relatively high basal levels of SLe^{a}. The failure to obtain a statistically significant upregulated expression of SLe^{a} in 22A-WT cells suggests that once a threshold of FX is produced by the tumor cell, a maximal biosynthesis/expression of this fucosylated glycan takes place.

6. Rolling of E48/FX^{hi} HNSCC on E-Selectin and on TNFα-Activated HUVEC

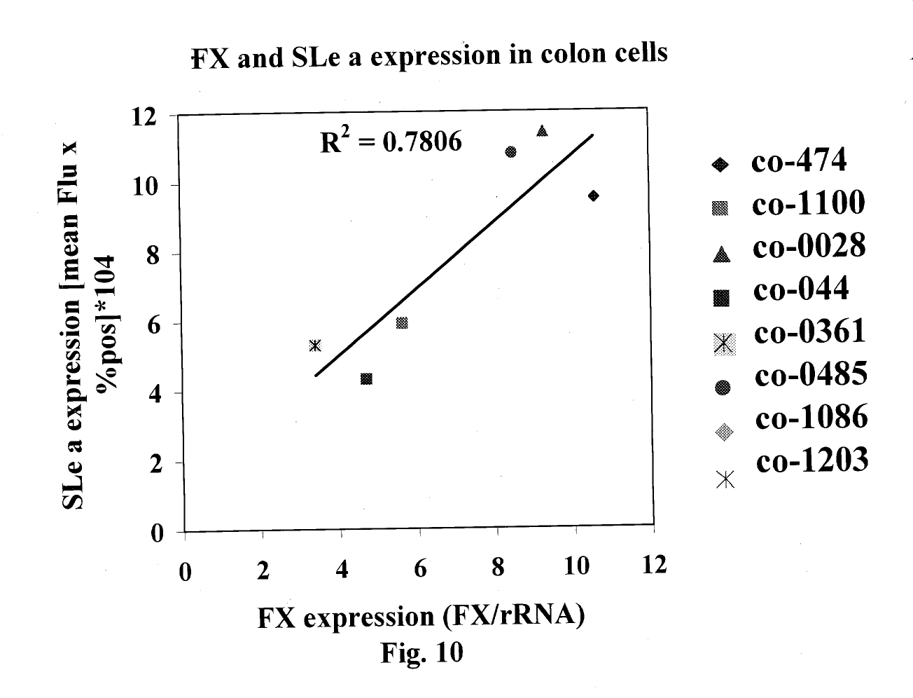

In order to test if the induction of SLe^{a} carbohydrate epitopes triggered by an upregulated FX expression in HNSCC cells is physiologically relevant for the ability of these cells to interact with a major vascular receptor for this ligand, E-selectin, compared in in vitro flow chamber assays the ability of high or low E48 expressing cells to tether to and roll on artificial substrates coated with recombinant E-selectin under physiological shear flow. When perfused over a plate coated with E-selectin at a shear stress of 1 dyn/cm², the lower range of physiological shear stressed found in post capillary venules in vivo, only high E48 cells but none of the low E48 cells could tether and roll on the adhesive substrate (FIG. 9A). All adhesive interactions were Ca²⁺ specific as they were eliminated in the presence of the Ca²⁺ chelator EDTA. Rolling adhesions were persistent at 1 dyn/cm² but weaker than those of neutrophils since 10 fold elevation of the shear stress enhanced the detachment of these cells but not of PMN from the E-selectin coated substrate (data not shown). Nevertheless, the majority of E48^{hi} cells which accumulated on E-selectin at low flow remained adherent and continued to roll on the selectin at medium shear stress range of 5 dyn/cm² (FIG. 9A). This adhesive capacity of E48^{hi} cells correlated well with their ability to form rolling adhesions on cytokine-stimulated vascular endothelial cells expressing E-selectin. When perfused over a monolayer of TNFα-activated HUVEC, only high E48^{hi} cells could tether and roll on the E-selectin expressing endothelial cells and the vast majority of these interactions could be specifically inhibited by E-selectin blocking MAb (FIG. 9B). This result indicates that E48^{hi} HNSCC express not only functional E-selectin ligands, but that these ligands determine almost exclusively their ability to initiate primary rolling adhesions on cytokine-stimulated HUVEC. Put together, these results demonstrate that E48^{hi} cells but not E48^{lo} cells express functionally adhesive E-selectin ligands and successfully use these ligands to tether and roll on vascular E-selectin under physiological shear flow.

7. Expression of FX in Cells Originating from Different Cancer Cell Lines

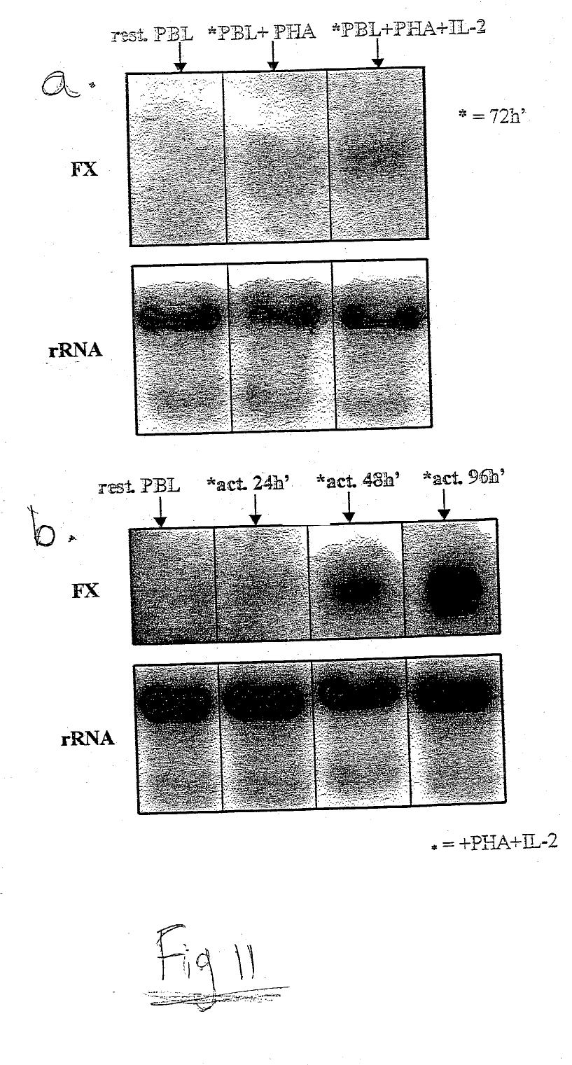

The level of FX mRNA expression and Sle^{a} expression in cells originating from nine different colon cancer cell lines was determined as explained above. As seen in FIG. 10, there was a positive correlation between the mRNA levels of FX and the expression of the Sle^{a} protein in the tested cells.

8. Expression of FX mRNA in Non Activated and Activated Leukocytes

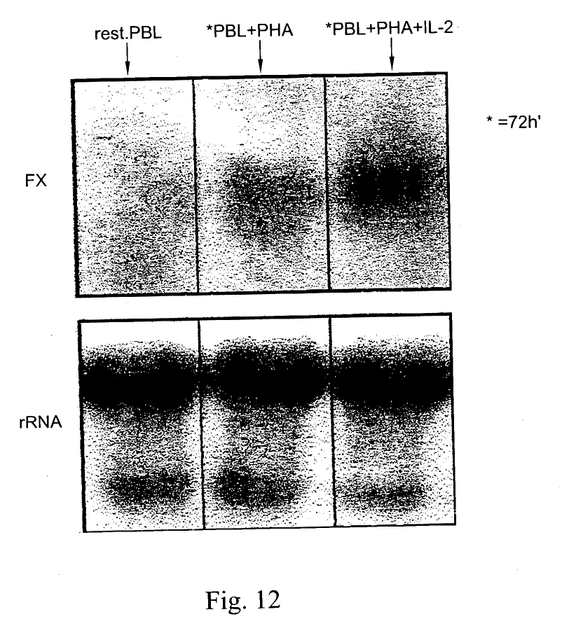

8.1 In order to determine the effect of activation of leukocytes on the level of expression of FX in the cells, peripheral blood mononuclear cells (PBMC) were activated either with PHA alone or with a combination of PHA and IL2. As seen in FIG. 11A, the level of FX mRNA expression was substantively elevated in PBL cells activated with a combination of PHA and IL2. The effect of the activation on the elevation of the level of FX mRNA was time dependent as can be seen in FIG. 11B.

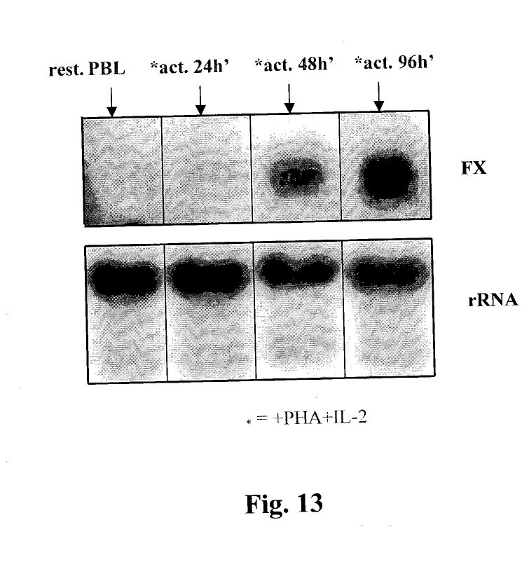

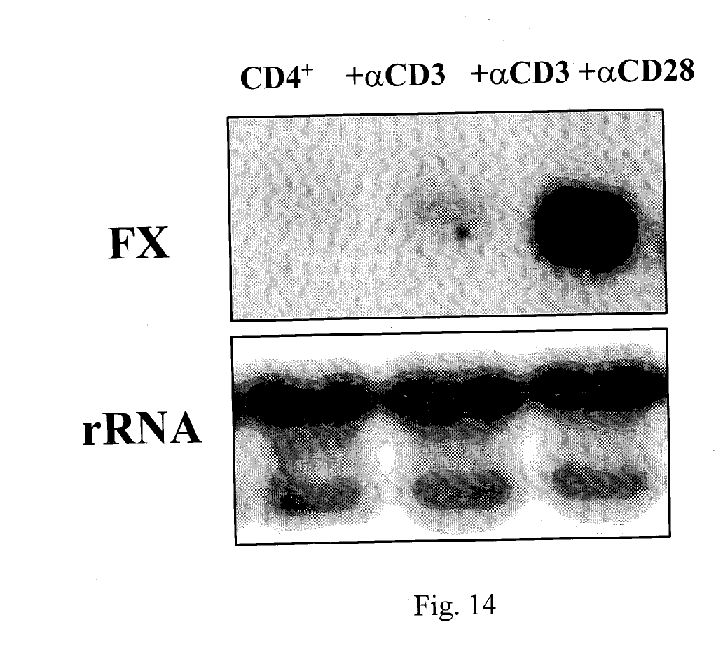

The effect of activation on the level of FX expression was tested in different kinds of lymphocytes. As seen in FIG. 12, activation of CD4 cells with anti-CD3 antibody (specific for T cell receptor) resulted in elevation in the level of FX mRNA levels in the CD4⁺ cells as compared to non activated CD4³⁰ cells. Activation of the CD4⁺ cells with a combination of anti-CD3 antibody and anti-CD28 antibody.

9. Mortality Rate of Nude Mice Injected with Cells Expressing High Levels of E48 or Low Levels of E48

The mortality rate of mice which were injected with cells expressing high levels of E48 was higher than that of mice injected with cells expressing low levels of E48. Mice of the former group began dying within about a month following injection of the cells while no mice belonging to the latter group died within five months following injection of the cells.

### SUMMARY

The above results generally show that there is an elevated expression of FX in both various types of cancer cells as well as in activated T cells. In some of the cases, there is a very high correlation between the level of expression of the FX and the level of expression of Sle^{a} (and in some cases Sle^{x}) proteins in these cells. The elevation in the level of FX expression in the cells was shown to be more prominent under certain conditions. In this case, activation in cancer cells was higher as a result of contact of the cells with an anti E48 antibody and the activation of T-cells was high following their contact with combinations of antibodies and cytokines. The high expression of FX correlated with the cells ability to adhere in vitro as well as to the metastic potential of the cancer cells. Thus, for the first time, these results show that it is possible to regulate the level of FX in cancer cells and activated leukocytes and by this, to effect the cells potential to adhere and metastasize.

1. A method for inhibiting or preventing adherence of cells to their target tissue or target cells comprising reducing the level of activity of FX in said cells.

2. A method according to claim 1, wherein said reduction is in the level of expression of FX protein in said cells.

3. A method according to claim 1, wherein said reduction is in the level of enzymatic activity of FX in said cells.

4. A method according to claim 1, wherein said cells are cancer cells.

5. A method according to claim 1, wherein said cells are leukocytes.

6. A method according to claim 5, wherein said leukocytes are T-cells.

7. A method according to claim 6, wherein said T-cells are CD4+ cells.

8. A method for inhibiting or preventing metastasis of cancer cells comprising reducing the level of activity of FX in said cancer cells.

9. A method according to claim 8, wherein said reduction is in the level of expression of FX protein in said cancer cells.

10. A method according to claim 8, wherein said reduction is in the level of enzymatic activity of FX in said cancer cells.

11. A method for inhibiting or preventing metastasis of cancer cells comprising contacting said cells with an effective amount of an agent which reduces the level of activity of FX in said cancer cells.

12. A method according to claim 8, wherein the reduction in activity of FX results in reduced expression of one or more selectin ligands on said cancer cells.

13. A method according to claim 11, wherein said agent is an extracellular agent.

14. A method according to claim 13, wherein said extracellular agent is an antibody which binds to an extracellular moiety on said cells and fractions or derivatives of said antibody which essentially maintain said antibody's binding characteristics.

15. A method according to claim 14, wherein said antibody is an anti-E48 antibody.

16. An agent which enhances the level of activity of FX in cancer cells.

17. An agent according to claim 16, wherein said enhanced level of activity is enhanced expression of FX protein in cancer cells.

18. A vaccine for use in the prevention or treatment of cancer comprising an effective amount of cancer cells expressing a high level of expression of FX protein.

19. A method for enhancing the immunogenicity of cancer cells comprising contacting said cells with an effective amount of an agent which enhances the level of FX in said cells.

20. A method for the inhibition or prevention of an undesired inflammatory response, an auto-immune process or transplant rejection involving activation of leukocytes; said method comprising reducing the level of activity of FX in said leukocytes.

21. A method according to claim 20, wherein said leukocytes are T-cells.

22. A method according to claim 21, wherein said T-cells are CD4⁺ T-cells.

23. A method for inhibition or prevention of an inflammatory response, an auto-immune process or transplant rejection comprising contacting leukocytes of a treated individual with an effective amount of an agent which inhibits or prevents the activity of FX in said leukocytes.

24. A method according to claim 23, wherein said inhibition or prevention of an inflammatory response results from inhibition of activation of FX by an activating factor in said leukocytes.

25. A method for enhancing a desired immune reaction comprising elevating the level of activity of FX in leukocytes involved in said reaction.

26. A method for determining the state of a malignant disease involving cancer cells comprising:
(a) analyzing the level of activity of FX in said cancer cells;
(b) comparing said level to the level of activity of FX in cancer cells being in different stages of the disease to find a level of activity of FX essentially equal to the level of activity of FX activity measured in (a) above and determining the stage of the malignant disease.

27. A method for determining the probability of formation of metastasis by cancer cells comprising:
(a) measuring the level of activity of FX in said cancer cells;
(b) comparing said level to the level of activity of FX in non-malignant cells, a measured level higher than that of the. level in non-malignant cells indicating a high probability that said cells will form metastasis.

28. A method according to claim 27, wherein said measured level of activity of FX in said cancer cells is compared to a predetermined threshold level, said threshold level being calculated on the basis of a number of measurements of FX activity in non-malignant cells, a measured level higher than the threshold level indicating a high probability that said cells will form metastasis.

29. A method for determining an individual's immune response to an immunogen comprising:
(a) administering said immunogen to the individual;
(b) determining the level of activity of FX in immunocytes of said individual;
(c) comparing the level of activity of FX in said cells to the level of activity of FX in immunocytes of at least one non-immunized individual which was not administered with said immunogen, a level of measured activity of FX higher than the level of activity of FX expression in immunocytes of said at least one non-immunized individual, indicating a high probability of an immune resposne in said individual.

30. A method according to claim 29, wherein the measured level of activity of FX in leukocytes of said immunized individual is compared to a predetermined threshold level, said threshold level being calculated on the basis of measurements in at least two non-immunized individuals which were not administered with said immunogen, a measured level of activity of FX higher than said threshold indicating a high probability of an immune response in said individual.
